(19) United States
(12) Patent Application Publication
(10) Pub. No.: US 2011/0288289 A1
(43) Pub. Date: Nov. 24, 2011

(54) Preparation of Carbapenem Intermediate and Their Use

(75) Inventors: Wei-Hong Tseng, Miao-Li County (TW); Wen-Hsin Chang, Miao-Li County (TW); Yuan-Liang Kuo, Miao-Li County (TW); Chia-Mao Chang, Miao-Li County (TW)

(73) Assignee: SAVIOR LIFETEC CORPORATION, Maio-Li County (TW)

(21) Appl. No.: 12/783,166

(22) Filed: May 19, 2010

(51) Int. Cl. C07D 477/26; C07D 205/12

(52) U.S. Cl. 540/350; 540/302

(57) ABSTRACT

The present invention relates to preparing carbapenem intermediates that are useful to produce Ertapenem, Meropenem and Doripenem.

### BACKGROUND OF THE INVENTION

1. Field of the Invention

The present invention relates to a novel processes for the preparation of carbapenem intermediates that are useful to produce amorphous Ertapenem, Meropenem and Doripenem.

2. The Prior Arts

The carebapenem are among the most broadly effective antibiotics making them useful in the treatment of a wide range of bacterial infections. The continuing emergence of bacteria exhibiting resistance to existing therapeutic agents has made development of new carbapenem an important part of our strategy in addressing this problem.

Ertapenem sodium of carbapenem antibiotics is commercially available as Invanz® from Merck, and has chemical name [4R,5S,6S]-3-[[(3S,5S)-5-[[(3-carboxyphenyl)amino]carbonyl]-3-pyrrolidinyl]-thio]-6-[(1R)-1-hydroxyethyl]-4-methyl-7-oxo-1-azabicyclo[3.2.0]hept-2-ene-2-carboxylic-acid. 1β-methylcarbapenem antibiotic is Ertapenem of formula (II) in the present invention, and used as antibiotic agent in the treatment of moderate to severe complicated foot infection due to indicated pathogens in diabetic patients without osteomyelitis, 1β-methylcarbapenem antibiotic is also useful in the treatment of pneumonia, urinary tract infections, intra-abdominal, gynecological, skin, and soft tissue infections, meningits, septicemia and febrile Neutrogena.

In view of the importance of 1β-methylcarbapenem antibiotic, several synthetic procedures to prepare the compound have been reported. U.S. Pat. No. 5,478,820 and U.S. Pat. No. 5,856,321 claim various processes for preparing Ertapenem and its sodium salt. Example 12 of U.S. Pat. No. 5,487,820 discloses a process in which the Ertapenem was isolated by using column purification as well as freeze-drying technique. U.S. Pat. No. 6,504,027 provides a process for preparing Ertapenem in crystalline form which comprises deprotecting and extracting a polar organic solution containing a crude mono-protected Ertapenem of formula:

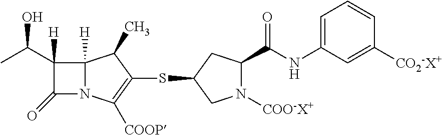

wherein P′ represents protecting group with C4-10 alcohol in the presence of ion-pairing reagent followed by a adjusting the pH to 5.5, collecting and crystallizing the resultant aqueous phase to produce a crystalline compound. Since this patent involves number of operations such as column chromatograph, expensive ion-pairing reagent and extraction. Therefore, this is an expensive and labor intensive technique.

However, there general problems with preparation of Ertapenem compounds such as occurrence of undesired by-products, complexity of synthesis, low yields, and subsequently high cost.

The aim of the present invention is to provide novel efficient processes of synthesizing some known Ertapenem compounds and to provide new intermediate compounds.

### SUMMARY OF THE INVENTION

Accordingly, the present invention provides a novel process for preparing compounds of formula II:

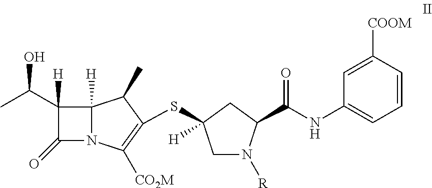

or a pharmaceutically acceptable salt thereof, wherein R is hydrogen or a protecting group and M is hydrogen or sodium ion, comprising: providing a compound of formula Ia:

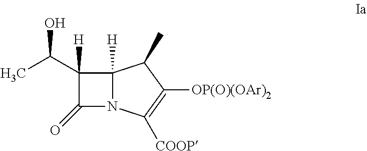

wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like.

Then the processes are performed by converting the compound of formula Ia into the compound of formula II, wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, and the like; and wherein the converting comprises the step of condensing the compound of formula Ia with a compound of formula V to form a compound of formula VP

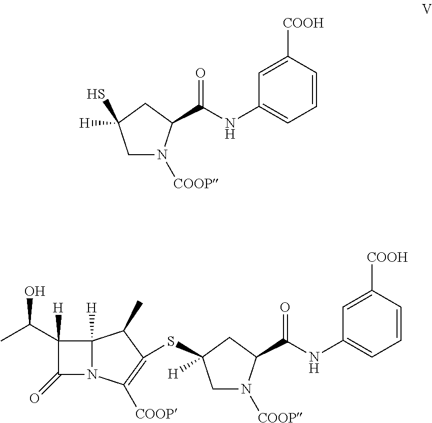

wherein P″ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like.

The present invention relates to formula Ia is a novel carbapenem intermediate, and formula II is 1β-methylcarbapenem antibiotic.

The objective of the present invention is to provide a simple, commercially viable, and industrially scalable process for the preparation of 1β-methylcarbapenem antibiotic, which avoids techniques like column chromatography with excellent yield and higher than 98% purity. And the present invention also provides a commercially viable and industrially scalable process for the preparation of Meropenem and Doripenem.

The details of one or more embodiments of the invention are set forth in the description below. Other features, objects, and advantages of the invention will be apparent from the description and from the claims.

### DETAILED DESCRIPTION OF THE PREFERRED EMBODIMENT

The 1β-methylcarbapenem antibiotic of formula II can be conveniently and economically prepared by reacting between formula I and formula V to obtain the compound of formula VI. The 1β-methylcarbapenem antibiotic of formula II is resulted from deprotecting the protecting group of formula VI in presence of a prereduced metal catalyst and a base according to the following reaction in Scheme 1:

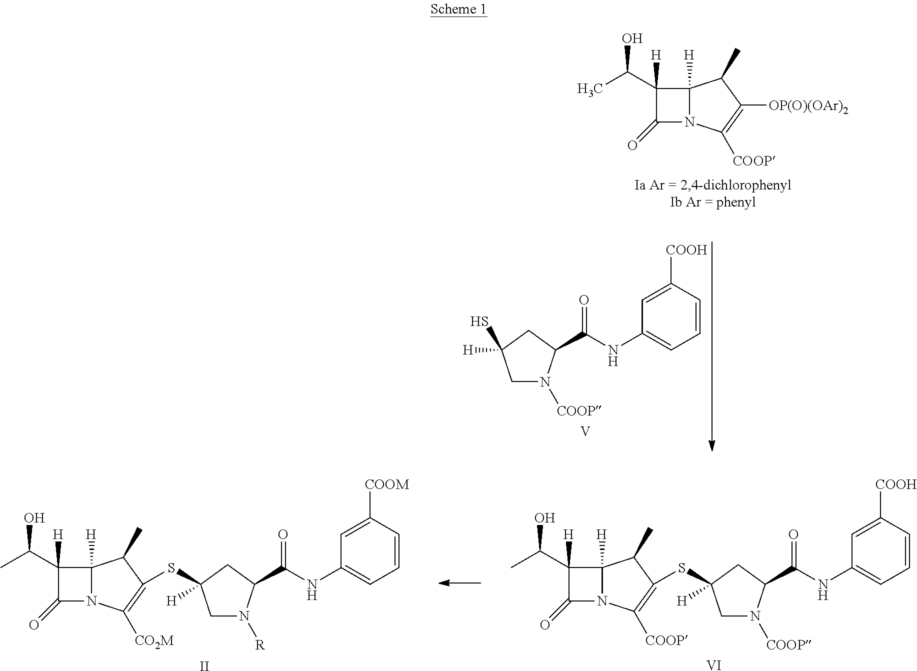

wherein Ar is 2,4-dichlorophenyl or phenyl, P is phosphor, and P′ and P″ are each independently selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like; or P′ and P″ are preferably p-nitrobenzyl; and wherein R is hydrogen or a protecting group such as carbenzyloxy or p-nitrobenzyl carbmoyl (PNZ) and M is hydrogen or sodium ion.

The compound of formula I is condensed with compound of formula V in the presence of a prereduced metal catalyst and a base and in the presence or absence of solvent to obtain the compound of formula VI, wherein the solvent is selected from the group consisting of diethyl ether, tetrahydrofuran, toluene, xylene, dichloromethane, 1,2-dichloroethane, N,N-dimethylformamide, dimethylacetamide, N-methylpyrrolidinone, N-ethylpyrrolidinone, N-methylpiperidinone, acetonitrile, propionitrile, and mixtures thereof, and wherein the prereduced metal catalyst is palladium, platinum or rhodium, and wherein the base is hydroxide or bicarbonate which was obtained by mixing carbon dioxide.

The compound of formula VI is deprotected its protecting group in the presence or absence of sodium ion source base and a solvent, and the pH of the aqueous layer is optionally adjusted. The reaction conditions for deprotection depend on the nature of the protecting groups utilized. For instance, the 2,2,2-trichloroethoxycarbonyl group is preferably removed by treatment with zinc and glacial acetic acid; the p-nitrobenzyloxycarbonyl and allyloxycarbonyl are removed by treating with hydrogen in the presence of a noble metal catalyst such as palladium or its complex. In another embodiment of the present invention, the solvent used for deprotection using hydrogenolysis is selected from THF, acetonitrile, dioxane, ethyl acetate, isopropyl alcohol, methanol, dichloromethane, DMF, base, water or mixtures thereof, and catalyst employed for reduction is selected from palladium on carbon, platinum, platinum oxide and the like. The deprotection of protecting groups can be carried out using a mixture of solvents either in single phase or in biphasic medium. The purification is treated with an activated carbon.

The volume of aqueous layer is condensed by the extraction of butanol or isoamyl alcohol (IAA) to obtain high concentration of formula II in aqueous layer. Finally, an alcohol is charged to the aqueous layer at a temperature in the range of −20° C. to 10° C., wherein the alcohol is selected from the group consisting of methanol, ethanol, 1-propanol, isopropyl alcohol tetrahydrofuran, and mixtures thereof; and 1β-methylcarbapenem antibiotic of formula II in amorphous form is isolated.

It is preferable to use equipment that is capable of multi-stage extraction such as centrifugal extractor for optimal performance. Most preferable is the use of a multi-stage centrifugal extractor. The preferred equipment is dependent on scale; CINC (Costner Industries Nevada Corporation) liquid-liquid centrifugal separators are preferred for laboratory scale operation; whereas, a Podbielniak® centrifugal extractor is preferred for large scale operation.

The invention relates to a process for the preparation of the novel compound of formula Ia as defined above.

For the purpose, a compound of formula III, wherein P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like, is cyclized with a rhodium catalyst in 4-Dicyanomethylene-2-methyl-6-p-dimethylaminostyryl-4H-pyran (DCM) at a temperature in the range of 30° C. to 60° C. to obtain a compound of formula IV. As used herein, the term “rhodium catalyst” refers to dimeric rhodium salts selected from the group consisting of rhodium octanoate Rh₂(Oct)₄, rhodium acetate Rh₂(Ac)₄, rhodium octanate Rh₂(HAc)₄ and rhodium trifluoroacetate Rh₂(O₂CCF₃)₄.

The compound of formula IV is reacted with bis(2,4-dichlorophenyl)-chlorophosphate (DDCP) or bisphenyl chlorophosphate (DPCP) in the presence of a base to obtain the compound of formula I shown in Scheme 2.

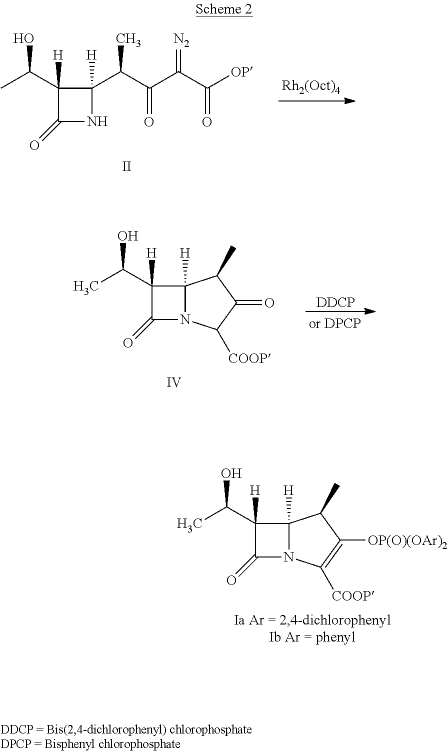

A process of cyclizing a compound of formula III is performed using a organic solvent selected from the group consisting of dichloromethane, methyl acetate, ethyl acetate, toluene, tetrahydrofuran, acetonitrile, and mixtures thereof. And preparation of formula I by reacting the compound of formula IV with bis(2,4-dichlorophenyl)-chlorophosphate (DDCP) or bisphenyl chlorophosphate (DPCP) in the presence of an organic base such as diisopropylethylamine (DIPEA), diisopropylamine (DIPA), dicyclohexylamine (DCHA), 2,2,6,6-tetra-methylpiperidine (TMP), 1,1,3,3-tetra-methylguanidine (TMG), 1,8-diazabicyclo[4.3.0.]undec-7-ene (DBU) 1,5-diazabicyclo[4.3.0]non-5-ene (DBN), N-methylpyrrolidine, N,N-dimethyl-aminopyridine, N,N-diethylamino pyridine potassium hydroxide, sodium carbonate, sodium hydrogen carbonate, or disodium hydrogen phosphate. The preferred organic base is diisopropylethylamine (DIPEA). To avoid impurity formation, the condensation reaction can be optionally conducted in a base like N,N-dimethyl aminopyridine, N,N-diethylamino pyridine.

Compounds of formula I as carbapenem intermediates can be used to prepare many of carbapenem antibiotics such as 1β-methylcarbapenem antibiotic, Meropenem, Doripenem, Ertapenem, etc. as shown in Scheme 3. The compound of formula Ib is a commercial product. The present invention provides a novel compound of formula Ia. The compound of formula Ia is reacted with sulfur side chain to obtain the protected carbapenem product with higher yield and purity than the compound of formula Ib.

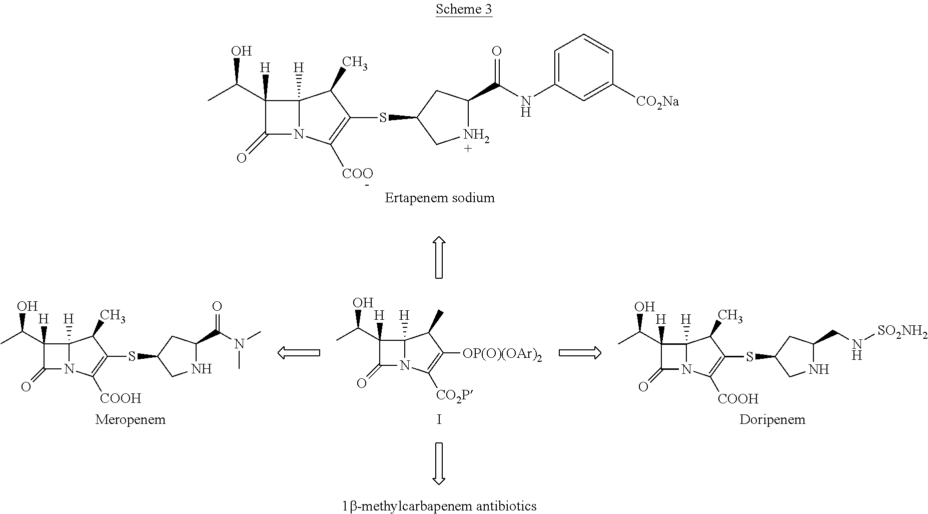

The formula I can be readily converted to Meropenem and Doripenem by one of several methods shown in Scheme 4. Meropenem prepared by reacting between formula I and formula VII to obtain the compound of formula VIII. Meropenem is resulted from deprotecting the protecting group of formula VIII in presence of a prereduced metal catalyst and a base. In addition, Doripenem prepared by reacting between formula I and formula IX to obtain the compound of formula X, wherein Ar is 2,4-dichlorophenyl. Doripenem is resulted from deprotecting the protecting group of formula X in presence of a prereduced metal catalyst and a base,

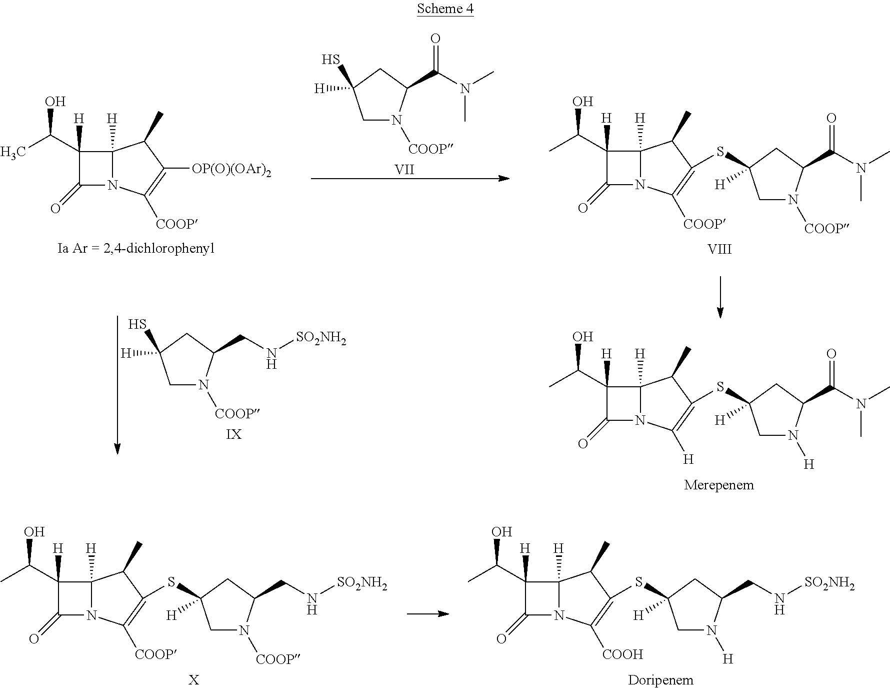

wherein Ar is 2,4-dichlorophenyl, P is phosphor, and P′ and P″ are each independently selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like; or P′ and P″ are preferably p-nitrobenzyl.

A further embodiment of the present invention concerns the use of the compound of formula I and processes of any of the preceeding claims for the preparation of a compound of formula I.

In addition, the present invention relates to the use of compound of formula I for the preparation of 1β-methylcarbapenem antibiotic, Meropenem and Doripenem which are commercially available.

Of the compounds, the invention relates especially to those of formulae I to X as such, especially those in which the substituents correspond to the radicals indicated in the respective Examples.

Numerous salt-forming ions are recited in Berge, S. M., et al. J. Pharm. Sci. 66(1): 1 16 (1977), the teachings of which are incorporated herein by reference. The charge balancing group X⁺ maintains overall charge neutrality. Preferably X⁺ represents a pharmaceutically acceptable salt-forming cation. Preferred salt-forming cations are selected from the group consisting of: sodium, potassium, calcium and magnesium. More preferably the salt-forming cation is a member selected from the group consisting of: Na⁺, Ca⁺² and K⁺.

Special preference is given to the compounds Ia, Ib mentioned in the Examples, especially each individual compound.

The present relates especially to the reaction steps and new intermediate compounds mentioned in the following Examples. Although the present invention has been described with reference to the preferred embodiments thereof, it is apparent to those skilled in the art that a variety of modifications and changes may be made without departing from the scope of the present invention which is intended to be defined by the appended claims.

### Example 1

### A) Preparation a Compound of Formula Ia

The compound of formula III (43.2 g) is slurried in DCM (777 ml). Followed by adding Rh₂Oct₄ (145 mg) to the solution. The mixture is heated at reflux for 7 hours then distills the resulting solution to remove 4-Dicyanomethylene-2-methyl-6-p-dimethylaminostyryl-4H-pyran (DCM, 388 mL). The compound of formula IV in dichloromethane solution is cooled to less than −35° C. The bis(2,4-dichlorophenyl) chlorophosphate (71.3 g) and the mixture of diisopropyl ethylamine (17.63 g) and 4-dimethylamino pyridine (40 mg) in DCM (43 ml) are added to the reaction solution at less than −35° C. The reaction solution is aged for 2 hours. Then extracted it by 1% HCl_{(aq)} (100 mL) and 5% NaHCO_{3(aq)} (100 mL) at 0˜5° C. The resulting dichloromethane solution is obtained as a compound of formula Ia and taken for Example 2A, 4A and 4B.

### B) Preparation a Compound of Formula Ib

The compound of formula III (43.2 g) is slurried in DCM (389 ml). Followed by adding Rh₂Oct₄ (130 mg) to the solution. The mixture is heated at reflux for 6 hours. The compound of formula IV in dichloromethane solution is cooled to less than −5° C. The bisphenyl chlorophosphate (32.7 g) and the mixture of diisopropyl ethylamine (18.6 g) and 4-dimethylamino pyridine (0.2 g) in DCM (43.2 ml) are added to the reaction solution at less than −5° C. The reaction solution is aged for 2 hours. Then extracted it by 1% HCl_{(aq)} (200 mL) and 5% NaHCO_{3(aq)} (200 mL) at 0˜5° C. and crystallized from ethyl acetate and heptanes. The resulting solid is obtained as a compound of formula Ib and taken for Example 2B.

### Example 2

### A) Preparation of a Compound of Formula VI from Formula Ia

To provide p-nitrobenzyl(1R,5S,6S)-6-[(IR)-1-hydroxyethyl]-2-[bis(2,4-dichlorophenyl)phosphono)oxy]-1-methylcarbapen-2-em-3-carboxylate of formula Ia in dichloromethane (500 mL) from Example 1A at −30° C., 3-([[(2S,4S)-mercapto-2-pyrrolidinyl-1-(4-nitrobenzyloxy)carbonyl]carbonyl]amino]benzoic acid (44.7 g) was added. To the reaction mixture, diisopropylethylamine (41.0 g) was added at −30° C. and stirred. After completion of reaction water (500 mL) were added to resulting mixture into, stirred, and separated. The organic layer was obtained to yield the compound of formula VI and taken for subsequent step described in Example 3A.

### B) Preparation of a Compound of Formula VI from formula Ib

To provide p-nitrobenzyl(1R,5S,6S)-6-[(IR)-1-hydroxyethyl]-2-[(di-phenylphosphono)oxy]-1-methylcarbapen-2-em-3-carboxylate of formula Ib (119 g) obtained from Example 1B in acetonitrile (560 g) at −5° C., 3-([[(2S,4S)-mercapto-2-pyrrolidinyl-1-(4-nitrobenzyloxy) carbonyl]carbonyl]amino]benzoic acid (90 g) was added. To the reaction mixture, diisopropylethylamine (72 g) was added at −10° C. and stirred. After completion of reaction the reaction solution was distilled to remove acetonitrile. Water (1.8 kg) and dichloromethane (1.6 kg) were added to resulting mixture into, stirred, and separated. The organic layer was obtained to yield the compound of formula VI and taken for subsequent step described in Example 3B.

### C) Preparation of a Compound of Formula VI from formula Ib

To provide p-nitrobenzyl(1R,5S,6S)-6-[(IR)-1-hydroxyethyl]-2-[(di-phenylphosphono)oxy]-1-methylcarbapen-2-em-3-carboxylate of formula Ib (119 g) purchased from SHILANG (Zhuoli Group)-Pharma (NANJING) CO., LTD in acetonitrile (560 g) at −5° C., 3-([[(2S,4S)-mercapto-2-pyrrolidinyl-1-(4-nitrobenzyloxy) carbonyl]carbonyl]amino]benzoic acid (90 g) was added. To the reaction mixture, diisopropylethylamine (72 g) was added at −10° C. and stirred. After completion of reaction the reaction solution was distilled to remove acetonitrile. Water (1.8 kg) and dichloromethane (1.6 kg) were added to resulting mixture into, stirred, and separated. The organic layer was obtained to yield the compound of formula VI and taken for subsequent step described in Example 3C.

### Example 3

### A) Preparation of a Compound of Formula II, R=H

The compound of formula VI in dichloromethane solution from Example 2A was added to the 10% Pd on carbon (48 g) with purified process water (648 mL) and sodium bicarbonate (37.2 g) at 20.0° C. Conduct hydrogen gas replacement twice for the nitrogen in the reactor. Then adjust the pressure from 25 psi to 80 psi during first one hour under hydrogen. Control reaction temperature was at 20° C. for 4˜5 hours. Cool down the reaction temperature to be less than 10° C. Then adjust the pH value to about 5.0 with 5% HCl. Filter 10% Pd/C out and separate organic layer. The pH value of the aqueous lay was adjust to be about 6.50 and then extract by adding dichloromethane (about 1 kg). n-Butanol (3 kg, 2 kg) is used to extract the resulting aqueous solution twice at 0˜5° C. The aqueous solution is filtered through 0.22 um filter. Isopropranol (66 ml) is added to concentrated aqueous solution (330 ml; ˜100 mg/ml) at −2 to 5° C. Then cool to −8 to 0° C. and charge the mixture of methanol and tetrahydrofuran (99 ml; 2/1; V/V). Adjust the pH value by 20% acetic acid in methanol to be about 5.7 at −8° C. to −5° C. The mixture solvent of IPA, methanol and tetrahydrofuran (148.5 ml; 4/2/1; V/V) is added to resulting solution again. Seed (0.7 g, etrapenem sodium; >98% purity) is added to the mixture and aged for 2 hour. The mixture solvent of IPA, methanol and tetrahydrofuran (346.5 ml; 0.15/2/1; V/V) is added to resulting solution at −5 to −15° C. and aged for more than 5 hours. Filter the solid, wash the wet cake by mixture solvent of methanol and tetrahydrofuran (60 ml; ½; V/V) and press with nitrogen to filtration to remove the solvents until LOD<13%. Yield product with 26.4 g, >98% purity, LOD=8%.

### B) Preparation of a Compound of formula II, R=H

The compound of formula VI in dichloromethane solution from Example 2B was added to the 10% Pd on carbon (71 g) with purified process water (1.2 kg) and sodium bicarbonate (67 g) at 20.0° C. Conduct hydrogen gas replacement twice for the nitrogen in the reactor. Then adjust the pressure from 25 psi to 80 psi during first one hour under hydrogen. Control reaction temperature was at 20° C. for 4˜5 hours. Cool down the reaction temperature to be less than 10° C. Then adjust the pH value to about 5.0 with 5% HCl. Filter 10% Pd/C out and separate organic layer. The pH value of the aqueous lay was adjusted to be about 6.50 and then extract by adding dichloromethane (about 1 kg). n-Butanol (7.5 kg, 4.5 kg) is used to extract the resulting aqueous solution twice at 0˜5° C. The aqueous solution is filtered through 0.22 um filter.

The mixture of methanol and tetrahydrofuran (250 ml; ½; V/V) is added to concentrated aqueous solution (500 ml; ˜100 mg/ml) at −2 to 5° C. Adjust the pH value by 20% acetic acid in methanol to be about 5.7 at −3 C to 0° C. The mixture solvent of methanol and tetrahydrofuran (250 ml; ½; V/V) is added to resulting solution again. Seed (1.0 g, etrapenem sodium; >98% purity) is added to the mixture at −8 to −5° C. and aged for 1 hour. The mixture solvent of methanol and tetrahydrofuran (1000 ml; ½; V/V) is added to resulting solution at −5 to −15° C. and aged for more than 5 hours. Filter the solid, wash the wet cake by mixture solvent of methanol and tetrahydrofuran (80 ml; ½; V/V) and press with nitrogen to filtration to remove the solvents until LOD<13%. Yield crude product with 56.0 g, >98% purity, LOD=7%.

The compound of formula Ia in reaction solution will be reacted with sulfur side chain directly without isolation to generate VI, VIII, or X with >90% purity (see Table 1). Followed by hydrogenation and crystallization to get the final products such as carbapenem, Meropenem, Doripenem, and Ertapenem.

TABLE 1

| Example | Intermediate | Yield(from III to II)* | Purity* |
|---|---|---|---|
| 3A | Formula Ia | 63.3% | 82.5% |
| 3B | Formula Ib | 60.2% | 79.3% |

*The yield and purity were obtained after hydrogenation.

### C.1) Preparation of a Compound of Formula II, R=H

The compound of formula VI in dichloromethane solution from Example 2C was added to the 10% Pd on carbon (71 g) with purified process water (1.2 kg) and sodium bicarbonate (67 g) at 20.0° C. Conduct hydrogen gas replacement twice for the nitrogen in the reactor. Then adjust the pressure from 25 psi to 80 psi during first one hour under hydrogen. Control reaction temperature was at 20° C. for 4˜5 hours. Cool down the reaction temperature to be less than 10° C. Then adjust the pH value to about 5.0 with 5% HCl. Filter 10% Pd/C out and separate organic layer. The pH value of the aqueous lay was adjusted to be about 6.50 and then extract by adding dichloromethane (about 1 kg). n-Butanol (7.5 kg, 4.5 kg) is used to extract the resulting aqueous solution twice at 0˜5° C. The aqueous solution is filtered through 0.22 um filter.

Isopropranol (100 ml) is added to the concentrated aqueous solution (500 ml; ˜100 mg/ml) at −2 to 5° C. Then cool to −8 to 0° C. and charge the mixture of methanol and tetrahydrofuran (150 ml; 2/1; V/V). Adjust the pH value by 20% acetic acid in methanol to be about 5.7 at −8° C. to −5° C. The mixture solvent of IPA, methanol and tetrahydrofuran (225 ml; 4/2/1; V/V) is added to resulting solution again. Seed (1.0 g, etrapenem sodium; >98% purity) is added to the mixture and aged for 1 hour. The mixture solvent of IPA, methanol and methyl acetate (525 ml; 0.15/2/1; V/V) is added to resulting solution at −5 to −15° C. and aged for more than 5 hours. Filter the solid, wash the wet cake by mixture solvent of methanol and methyl acetate (80 ml; ½; V/V) and press with nitrogen to filtration to remove the solvents until LOD<13%. Yield crude product with 54.8 g, >98% purity, LOD=11%.

### C.2) Preparation of a Compound of Formula II, R=H

The compound of formula VI in dichloromethane solution from Example 2C was added to the 10% Pd on carbon (71 g) with purified process water (1.2 kg) and sodium bicarbonate (67 g) at 20.0° C. Conduct hydrogen gas replacement twice for the nitrogen in the reactor. Then adjust the pressure from 25 psi to 80 psi during first one hour under hydrogen. Control reaction temperature was at 20° C. for 4˜5 hours. Cool down the reaction temperature to be less than 10° C. Then adjust pH value to about 5.0 with 5% HCl. Filter 10% Pd/C out and separate organic layer. The pH value of the aqueous lay was adjusted to be about 6.50 and then extract by adding dichloromethane (about 1 kg). n-Butanol (7.5 kg, 4.5 kg) is used to extract the resulting aqueous solution twice at 0˜5° C. The aqueous solution is filtered through 0.22 um filter. Isopropranol (100 ml) is added to the concentrated aqueous solution (500 ml; ˜100 mg/ml) at −2 to 5° C. Then cool to −8 to 0° C. and charge the mixture of methanol and tetrahydrofuran (150 ml; 2/1; V/V). Adjust the pH value by 20% acetic acid in methanol to be about 5.7 at −8° C. to −5° C. The mixture solvent of IPA, methanol and tetrahydrofuran (225 ml; 4/2/1; V/V) is added to resulting solution again. Seed (1.0 g, etrapenem sodium; >98% purity) is added to the mixture and aged for 1 hour.

The mixture solvent of 1-propanol, methanol and tetrahydrofuran (525 ml; 0.15/2/1; V/V) is added to resulting solution at −5 to −15° C. and aged for more than 5 hours. Filter the solid, wash the wet cake by mixture solvent of 1-propanol, methanol and tetrahydrofuran (80 ml; 1/1/2; V/V) and press with nitrogen to filtration to remove the solvents until LOD<13%. Yield product with 56.1 g, >98% purity, LOD=10%.

### C.3) Preparation of a Compound of Formula II, R=H

The compound of formula VI in dichloromethane solution from Example 2C was added to the 10% Pd on carbon (71 g) with purified process water (1.2 kg) and sodium bicarbonate (67 g) at 20.0° C. Conduct hydrogen gas replacement twice for the nitrogen in the reactor. Then adjust the pressure from 25 psi to 80 psi during first one hour under hydrogen. Control reaction temperature was at 20° C. for 4˜5 hours. Cool down the reaction temperature to be less than 10° C. Then adjust pH value to about 5.0 with 5% HCl. Filter 10% Pd/C out and separate organic layer. The pH value of the aqueous lay was adjust to be about 6.50 and then extract by adding dichloromethane (about 1 kg).

Iso-amyl alcohol (12 kg, 8 kg) is used to extract the resulting aqueous solution twice at 0-5° C. The aqueous solution is filtered through 0.22 um filter. Isopropranol (100 ml) is added to concentrated aqueous solution (500 ml; ˜100 mg/ml) at −2 to 5° C. Then cool to −8 to 0° C. and charge the mixture of methanol and tetrahydrofuran (150 ml; 2/1; V/V). Adjust the pH value by 20% acetic acid in methanol to be about 5.7 at −8° C. to −5° C. The mixture solvent of IPA, methanol and tetrahydrofuran (225 ml; 4/2/1; V/V) is added to resulting solution again. Seed (1.0 g, etrapenem sodium; >98% purity) is added to the mixture and aged for 1 hour. The mixture solvent of IPA, methanol and tetrahydrofuran (525 ml; 0.15/2/1; V/V) is added to resulting solution at −5 to −15° C. and aged for more than 5 hours. Filter the solid, wash the wet cake by mixture solvent of methanol and tetrahydrofuran (80 ml; ½; V/V) and press with nitrogen to filtration to remove the solvents until LOD<13%. Yield product with 55.8 g, >98% purity, LOD=10%.

### Example 4

### A) Preparation of a Compound of Formula VIII

To provide p-nitrobenzyl(1R,5S,6S)-6-[(IR)-1-hydroxyethyl]-2-[(bis(2,4-dichlorophenyl)phosphono)oxy]-1-methylcarbapen-2-em-3-carboxylate of formula Ia in dichloromethane (500 mL) from Example 1A at −35° C., (25,45)-2-(dimethyl-aminocarbonyl)-4-mercapto-1-(p-nitrobenzyloxycarbonyl)-1-pyrrolidine (33.6 g) was added. To the reaction mixture, diisopropylethylamine (32.3 g) was added at −30° C. and stirred. After completion of reaction, the resulting mixture was washed with 5% NaHCO₃ aqueous (500 ml) and water (500 ml) and separated. The organic layer was obtained to yield the compound of formula VIII and taken for subsequent step described in Example 5A.

### B) Preparation of a Compound of formula X

To provide p-nitrobenzyl(1R,5S,6S)-6-[(IR)-1-hydroxyethyl]-2-[(bis(2,4-dichlorophenyl)phosphono)oxy]-1-methylcarbapen-2-em-3-carboxylate of formula Ia in dichloromethane (500 mL) from Example 1A at −35° C., (2S,4S)-1-p-nitrobenzyloxycarbonyl-2-sulfamoylaminomethyl-4-mercaptopyrrolidine (39.0 g) was added. To the reaction mixture, diisopropylethylamine (32.3 g) was added at −30° C. and stirred. After completion of reaction, the resulting mixture was washed with 5% NaHCO₃ aqueous (500 ml) and water (500 ml) and separated. The organic layer was obtained to yield the compound of formula X and taken for subsequent step described in Example 5B.

### Example 5

### A) Preparation of Meropenem

The compound of formula VIII in dichloromethane solution from Example 4A was distilled to remove dichloromethane. Add tetrahydrofuran (1000 mL) into the condensed solution and stir until the complete dissolution. Deionized water (800 mL), 10% wt % Pd/C (8.0 g), and 2,6-dimethylpyridine (22 g) were added to the solution. The suspension was stirred at 20˜25° C. for 1 to 2 h under a H₂ atmosphere (1.8 MPa). The used Pd/C was removed by filtration and washed with a mixture of tetrahydrofuran (72 mL) and deionized water (48 mL). The filtrate was diluted with acetone (3200 mL) and seed crystals were added at 5 to 10° C. After 0.5 h, substantive crystals were precipitated. Acetone (1600 mL) was added slowly at 5 to 10° C. After the mixture was stirred for 1 h, the crystals were collected by filtration, washed with acetone (150 mL) and dried to give Meropenem 20.8 g.

### B) Preparation of Doripenem

The compound of formula X in dichloromethane solution from Example 4B was distilled to remove dichloromethane and dissolved in tetrahydrofuran (470 ml). Deionized water (310 mL), 10% wt % Pd/C (39.8 g), and MgCl₂.6H₂O (11.1 g) were added to the solution. The suspension was stirred from 25 to 35° C. for 2 to 3 h under a H₂ atmosphere (0.5 MPa). The used Pd/C was removed by filtration and washed with a mixture of tetrahydrofuran (140 mL) and deionized water (95 mL). MgCl₂.6H₂O (5.5 g) was dissolved in the combined filtrates. After addition of tetrahydrofuran (2300 mL) to the mixture, the aqueous layer was separated at 23˜28° C. After cooling the extract to 0˜5° C., MeOH (310 mL) and seed crystals (0.1 g) were added to the extract. After MgCl₂.6H₂O (5.5 g×2) was added to the organic layer, the resulting aqueous layer was separated and added to the previous aqueous suspension. MeOH (590 mL) was added dropwise to the suspension. The mixture was stirred at −10˜15° C. for 2 h. The solid was collected by filtration, washed with MeOH, and dried to give Doripenem 29.5 g.

A number of embodiments of the invention have been described. Nevertheless, it will be understood that various modifications may be made without departing from the spirit and scope of the invention. Accordingly, other embodiments are within the scope of the following claims.

What is claimed is:

1. A compound of formula Ia: 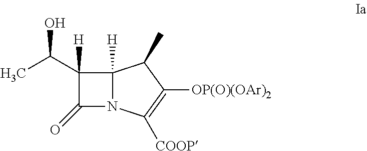
wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like.

2. The compound according to claim 1, wherein P′ is p-nitrobenzyl.

3. A process for the preparation of a compound of formula II: 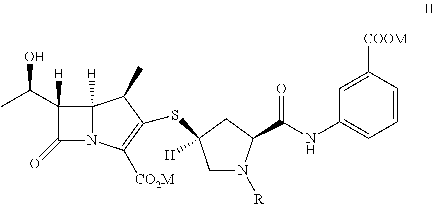
or a pharmaceutically acceptable salt thereof, wherein R is hydrogen or a protecting group and M is hydrogen or sodium ion; comprising:
i) providing a compound of formula Ia: 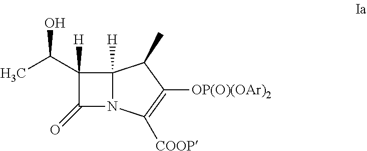
ii) converting the compound of formula Ia into the compound of formula II, wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like; and wherein the converting comprises the step of condensing the compound of formula Ia with a compound of formula V: 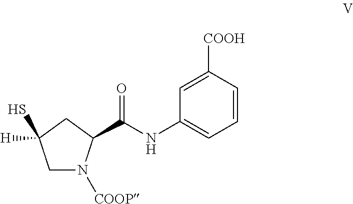
to form a compound of formula VI: 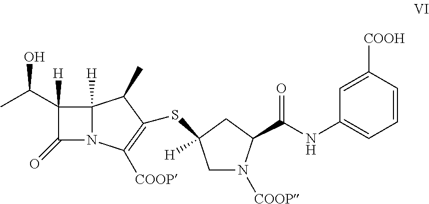
wherein P″ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, and the like.

4. The process according to claim 3, wherein R and M of the compound of formula II are hydrogen.

5. The process according to claim 3, wherein R of the compound of formula II is hydrogen and M of the compound of formula II is sodium ion.

6. The process according to claim 3, wherein the protecting group is carbenzyloxy or p-nitrobenzyl carbmoyl (PNZ).

7. The process according to claim 3, wherein the compound of formula Ia is converted into the compound of formula VI by using a solvent selected from the group consisting of diethyl ether, tetrahydrofuran, toluene, xylene, dichloromethane, 1,2-dichloroethane, N,N-dimethylformamide, dimethylacetamide, N-methylpyrrolidinone, N-ethylpyrrolidinone, N-methylpiperidinone, acetonitrile, propionitrile, and mixtures thereof.

8. The process according to claim 3, wherein the compound of formula VI is converted into the compound of formula II by hydrogenolysis in presence of a prereduced metal catalyst and a base, purifying and isolating with an alcohol to obtain the compound of formula II.

9. The process according to claim 8, wherein the prereduced metal catalyst is palladium, platinum or rhodium.

10. The process according to claim 8, wherein the base is hydroxide or bicarbonate which was obtained by mixing carbon dioxide.

11. The process according to claim 8, wherein the purification is treated with an activated carbon.

12. The process according to claim 8, wherein the isolation is conducted using a multi-stage countercurrent centrifugal extractor.

13. The process according to claim 8, wherein the alcohol is selected from the group consisting of methanol, ethanol, 1-propanol, isopropyl alcohol, n-butanol, tetrahydrofuran, and mixtures thereof.

14. The process according to claim 3, wherein the compound of formula Ia is prepared by a process comprising:
i) cyclizing a compound of formula III: 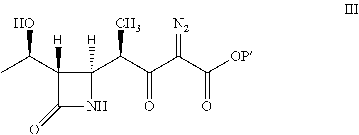
with Rh₂(Oct₄) in 4-Dicyanomethylene-2-methyl-6-p-dimethylaminostyryl-4H-pyran (DCM) to obtain a compound of formula IV: 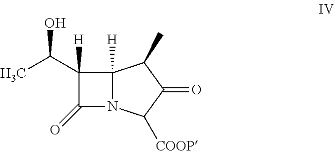
ii) reacting the compound of formula IV with bis(2,4-dichlorophenyl)-chlorophosphate in the presence of a base to obtain the compound of formula Ia.

15. The process according to claim 14, wherein the step (i) is performed using a solvent selected from the group consisting of dichloromethane, methyl acetate, ethyl acetate, toluene, tetrahydrofuran, acetonitrile, and mixtures thereof.

16. The process according claim 14, wherein step (ii) is performed using a base selected from the group consisting of diisopropylethylamine (DIPEA), diisopropylamine (DIPA), dicyclohexylamine (DCHA), 2,2,6,6-tetra-methylpiperidine (TMP), 1,1,3,3-tetramethylguanidine (TMG), 1,8-diazabicyclo[4.3.0.]undec-7-ene (DBU) 1,5-diazabicyclo[4.3.0]non-5-ene (DBN), N-methylpyrrolidine, N,N-dimethylaminopyridine, N,N-diethylamino pyridine potassium hydroxide, sodium carbonate, sodium hydrogen carbonate, and disodium hydrogen phosphate.

17. The process according claim 14, wherein P′ is p-nitrobenzyl.

18. A process for the preparation of a compound of Meropenem: 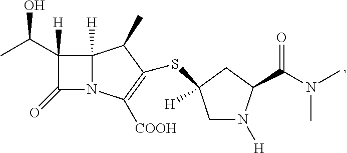
comprising:
i) providing a compound of formula Ia: 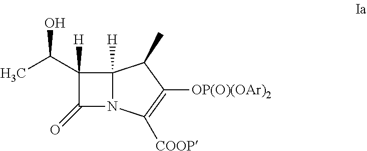
ii) converting the compound of formula Ia into Meropenem, wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like; and wherein the converting comprises the step of condensing the compound of formula Ia with a compound of formula VII: 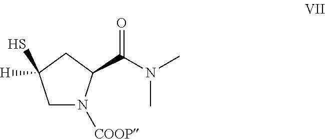
to form a compound of formula VIII: 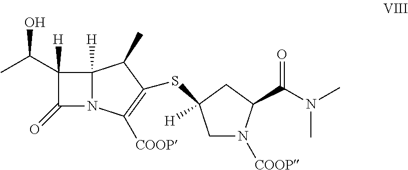
wherein P″ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, and the like.

19. A process for the preparation of a compound of Doripenem: 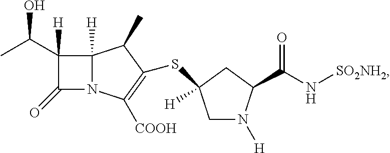
comprising:
i) providing a compound of formula Ia: 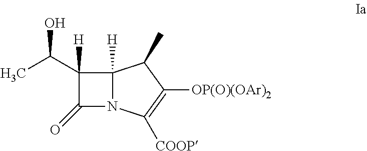
ii) converting the compound of formula Ia into Doripenem, wherein Ar is 2,4-dichlorophenyl, P is phosphor, and P′ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, hydrogen, and the like; and wherein the converting comprises the step of condensing the compound of formula Ia with a compound of formula IX: 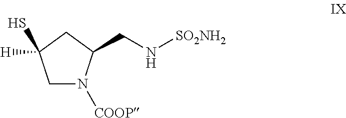
to form a compound of formula X: 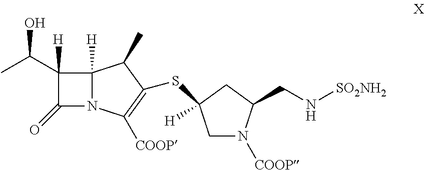
wherein P″ is selected from the group consisting of allyl, 2,2,2,-trichloroethyl, 2-bromoethyl, benzhydryl, trityl, aryl, trimethysilyl, triethylsil, 4-methoxybenzyl, t-butyl, p-nitrobenzyl, and the like.
